(19) United States
(12) Patent Application Publication
(10) Pub. No.: US 2002/0010157 A1
(43) Pub. Date: Jan. 24, 2002

(54) Composition and method for controlling insect pests and diseases on plants

(76) Inventors: Yousheng Duan, Zibo (CN); Zhiqiang Ju, Zibo (CN); Liye Ju, Zibo (CN); Zhiguo Ju, Wenatchee, WA (US)

Correspondence Address: ZHIGUO JU, 1229 4th St. # B., Wenatchee, WA, 98801, US

(21) Appl. No.: 09/860,994

(22) Filed: May 17, 2001

Related U.S. Application Data
(60) Provisional application No. 60/205,920, filed on May 22, 2000.

(51) Int. Cl. A61K 31/7032; A61K 31/685; A01N 43/04; A01N 57/26; A01N 37/02

(52) U.S. Cl. 514/78; 514/23; 514/552

(57) ABSTRACT

A composition and method for controlling diseases and pests on plants is provided. Specifically, the composition provides that polar glycerides selected from the group comprising monoglycerides, diglycerides, phospholipids, lysophospholipids, glycolipids, lysoglycolipids, or their derivatives control certain diseases or insect pests on plants.

### REFERENCE TO PRIOR APPLICATIONS

This application claims benefit of U.S. Provisional Application No. 60/205,920, filed May 22, 2000.

### BACKGROUND OF THE INVENTION

This invention pertains generally to the fields of pest and disease control on plants. Lipids, glyceride compositions more specifically, are provided for these applications.

Lipids represent a variety of compounds such as fatty acids, terpenoids, waxes, and glycerides. Glycerides are esters formed from glycerol and fatty acids, which include triglycerides, diglycerdes, monoglycerides, phospholipids, and glycolipids. The major components of most plant oils and animal oils or fats are triglycerides. Triglycerides do not have free polar group in their structure and therefore, are neutral glycerides. Monoglycerides, diglycerides, phospholipids, glycolipids, or their derivatives have free polar groups in their structure and therefore, are referred to as polar glycerides in the present invention.

Plant oils (triglycerides) or fatty acid soaps have been used for controlling pests in stored seeds and foods for hundreds of years (Giga and Munesti, 1990; Hill and Schoonhoven, 1981; Ran et al., 1988; Salas, 1985; Salas and Hernandez, 1985). Plant oils and fatty acid soaps are also tested for controlling pests on crops such as apples (Pless et al., 1995; Rock and Crabtree, 1987), grapes (Taschenberg, 1952), sweetpotatoes, lettuce, and cotton (Butler et al., 1988, 1991, 1993; Butler and Henneberry, 1989, 1990, 1991a, 1991b; Hesler and Plapp, 1986). U.S. Pat. No. 5,631,290 discloses the use of fatty acid salt as pesticides against soft-bodied insects and mites. In the United States, essential oil (terpenoids), jojoba oil (wax), canola oil (triglycerides), soybean oil (triglycerides), and fatty acid salts are registered as pesticides on certain plants (US EPA, 1992a, 1992b). However, the use of polar glycerides in controlling pests has not been disclosed before.

Lipids have also been used for disease control. The use of fatty acids soaps as cleaning and disinfecting agents can be traced to as early as 4000 years ago (Kabara, 1993). Neem seed oil possesses fungicidal activities (Locke, 1986, 1990) and reduces fruit decay in apple (Moline and Locke, 1993). When applied to trees in the fields, sunflower, olive, canola, corn, soybean, and grapeseed oil at 1% are effective in controlling apple powdery mildew (Podosphaera leucotrica), but canola and soybean oils at 1% have no effect on brown rot of peaches or black knot (Apiosporina morbosa) on leaves of plum and cherry (Northover and Schneider, 1991 and 1993; Northover and McFadden-Smith, 1995). U.S. Pat. Nos. 5,366,995, 6,103,768, and 6,136,856 discloses that fatty acid salts act as antifungal and antibacterial agents in plants. However, the antifungal activity of fatty acids has been controversial. While a C18:1 fatty acid salt gives moderate preventive control of two foliage plant diseases, it exacerbates two other diseases at the same time (Chase et al., 1983). And U.S. EPA (1992a) ruled that soap salts of fatty acids had no independent pesticidal activity in antimicrobial products (used on agricultural crops), and must be classified as inert ingredients in those products. Therefore, living plant was a different system from dead plant tissues.

The prior art teaches that esterafication of one molecule of fatty acid with one molecule of glycerol often results higher antimicrobial activity compared with the corresponding fatty acid while esterafication of three molecules of fatty acid with one molecule of glycerol reduces the antimicrobial activity of the corresponding fatty acid (Kabara, 1993). U.S. Pat. No. 4,002,775 discloses that monolaurin is the most effective antimicrobial agent among the fatty acid esters. Both monoglycerides (U.S. Pat. Nos. 4,997,851; 5,343,182) and phospholipids (U.S. Pat. No. 6,165,997) display antimicrobial activities. U.S. Pat. No. 6,033,705 teaches the use of monoglycerides and lysophospholipids for preventing microbial activity in foodstuffs such as meat and dehydrated fruit and vegetables. However, all these polar glycerides in the prior art were applied to either food or dehydrated fruit and vegetables, on which (dead foodstuffs) high concentrations of these polar glycerides can be used. The efficacy of these polar glycerides in controlling diseases on living plants and the possible phytotoxicity of these compounds to living plants have not been studied. In addition, the antimicrobial activity of other polar glycerides such as diglycerides or glycolipids, which share similar structure and properties with monoglycerides, in controlling plant disease is not known.

The inventors of the present compounds found that at the same concentration, polar glycerides selected from the group comprising mono- and diglycerides, phospholipids, glycolipids, or their derivatives were more effective than neutral glycerides such as triacylglycerols, plant oil, animal oil or fats in controlling certain diseases and pests on plants. Therefore, the objective of this invention is to provide a composition and a method to control insect pests and diseases on plants.

### SUMMARY OF INVENTION

The inventors of the present invention found that polar glycerides selected from the group comprising monoglycerides, diglycerides, phospholipids, lysophospholipids, glycolipids, lysoglycolipids, or their derivatives control certain diseases or pests on plants. The inventors of the present invention also found that said polar glycerides were more effective than neutral lipids such as triacylglycerols, plant oils, and animal oils or fats in controlling diseases or pests on plants.

Accordingly, the inventors of this invention provide a formulation and a method to make an emulsion with these polar glycerides as active ingredients. The emulsion comprises a polar glyceride or a mixture of polar glycerides, a neutral glycerides or a mixture of neutral glycerides, a surfactant, and water. The polar glycerides are selected from the group comprising monoglycerides, diglycerides, phospholipids, lysophospholipids, glycolipids, lysoglycolipids, their derivatives, or their mixture with any ratio. The neutral glycerides comprise triglycerides, glyceride type of plant oils, animal oils or fats, hydrogenated plant or animal oils, their derivatives, or their mixture with any ratio. The presence of neutral glycerides in the formulation is to reduce the phytotoxicity of the polar glycerides and is optional. And the surfactant includes any type of siloxane, polysiloxane, or non-ionic surfactants. The surfactant is also optional in the formulation.

According to the method of this invention, the polar glycerides comprise from approximately 1% up to 90% of the total emulsion, by volume; the neutral glycerides comprises from 0 to 50% of the total emulsion, by volume; the surfactant comprises approximately 0 to 10% of the total emulsion, by volume; and water comprises from 0 to 99.5% of the total emulsion, by volume. The emulsion can be prepared by different methods including (1) mixing the polar glycerides, neutral glycerides, and the surfactant to form a glyceride concentrate and diluting the concentrate with water to form a emulsion and (2) mixing the polar glycerides, neutral glycerides, surfactant, and water to form an emulsion.

According to the method of this invention, emulsions of polar glycerides are diluted with water at application. Emulsions can be used during dormant stage or during plant growing period. For dormant spray, emulsions containing 1% to 10% active ingredients, preferably 2.5% to 10% active ingredients, are applied on plants for disease or pest control. During plant growing season, emulsions containing 0.1% to 5%, preferably 0.5 to 3%, are applied to plants. The emulsion can be applied by any of the methods typically known and used in the agricultural industry for the application of a chemical. Preferably, the emulsion is applied by any common spraying technique used in the agricultural industry.

According to the present invention, plants may be treated include, but are not limited to, agricultural crops, fruit trees, vegetables, forage, bedding plants, nursery plants, ornamentals, houseplants, and turf grasses.

According to the present invention, the glyceride emulsions control, but are not limited to, the following pests: aphids, mites, pear psylla, scales, whiteflies, or other soft body pests or eggs of other pests.

According to the present invention, the glyceride emulsions control, but are not limited to, the following diseases caused by fungus, bacteria, and virus: powdery mildew, brown rot, wilt, bacterial canker, bacterial blight, scab, tobacco mosaic virus (TMV I), tomato mosaic virus (TMV II), tomato infectious chlorosis virus (TICV), tomato spotted wilt virus (TSWV), etc.

### DETAILED DESCRIPTION OF SPECIFIC EMBODIMENTS

(1) Definitions

The term ‘pest’, as used herein, refers to insects harmful to plants.

The term ‘neutral glycerides’, as used herein, refers to triglycerides or oils and fats (plant or animal originated) that contain triglycerides as major components.

The term ‘polar glycerides’, as used herein, refers to any glyceride that contains at least one free polar group in the structure.

The term ‘emulsion’, as used herein, refers to a stable mixture of two or more immiscibles held in suspension.

The term ‘surfactant’, as used herein, generally refers to surface active compounds which reduce surface tension when dissolved in water or a water solution, or which reduce interfacial tensions between two liquids.

The term ‘non-ionic surfactant’, as used herein, refers to surfactants that do not ionize in water and thus are not subject to hydrolysis by aqueous solutions of acid or alkali.

(2) Description

The inventors of the present invention found that polar glycerides selected from the group comprising monoglycerides, diglycerides, phospholipids, lysophospholipids, glycolipids, lysoglycolipids, or their derivatives control certain diseases or insect pests on plants. The inventors of the present invention also found that said polar glycerides were more effective than neutral glycerides such as triacylglycerols, plant oils, and animal oils or fats in controlling diseases or pests on plants.

Accordingly, the inventors of this invention provide a formulation and a method to make an emulsion with these polar glycerides as active ingredients. The emulsion comprises a polar glyceride or a mixture of polar glycerides, a neutral glyceride or a mixture of neutral glycerides, a surfactant, and water. The polar glyceride are selected from the group comprising monoglycerides, diglycerides, phospholipids, lysophospholipids, glycolipids, lysoglycolipids, their derivatives, or their mixture with any ratio. The neutral glycerides comprise triglycerides, glyceride type of plant oils, animal oils or fats, hydrogenated plant or animal oils, their derivatives, or their mixture with any ratio. The presence of neutral glycerides in the formulation is to reduce the phytotoxicity of the polar glycerides and is optional. And the surfactants include any type of siloxane, polysiloxane, or non-ionic surfactants. Examples of such surfactants are: polyether-polymethylsiloxane-copolymer (Break-Thru. RTM. OE441), manufactured by Goldschmidt Chemical Corporation; polyoxyethylenesorbitan, presently sold as the product family TWEEN and marketed by ICI Americas, Inc., of Wilmington, Del.; polyoxyethylene ethers, such as t-octylphenoxy-polyethanol, presently sold as the product family TRITON and marketed by Union Carbide Chemical and Plastics Co., Inc., of Danbury, Conn.; or alkylaryl polyoxyethylene glycols and alcohol, presently sold as Latron AG-98. Alternative surfactants with equivalent action to these typical products are also considered for use with the emulsion compound of the present invention. The use of surfactant is also optional in the formulation.

According to the method of this invention, the polar glycerides comprise from approximately 1% up to 90% of the total emulsion, by volume; the neutral glycerides comprises from 0 to 50% of the total emulsion, by volume; the surfactant comprises approximately 0 to 10% of the total emulsion, by volume; and water comprises from 0 to 99.5% of the total emulsion, by volume. The emulsion can be prepared by different methods including (1) mixing the polar glycerides, neutral glycerides, and surfactant to form a glyceride concentrate and diluting the mixture with water to form a emulsion at application and (2) mixing the polar glycerides, neutral glycerides, surfactant, and water to form an emulsion, which includes mixing the polar glycerides, neutral glycerides, and surfactant, heating the mixture to 50 to 90 degrees C., and adding hot water (pre-heated to 50 to 90 degrees C.) to the mixture with stirring. The mixture is left at room temperature for slow cooling. The emulsion thus formed is further diluted with water at application.

According to the present invention, plants may be treated include, but are not limited to, agricultural crops, fruit trees, and vegetables. It can also be used to control insect pests and diseases in forage, bedding plants, nursery plants, ornamentals, houseplants, and turf grasses.

According to the present invention, the glyceride emulsion controls but are not limited to the following pests: aphids, mites, pear psylla, scales, whiteflies, or other soft body pests or eggs of other pests.

According to the present invention, the glyceride emulsion controls but are not limited to the following diseases caused by fungus, bacteria, and virus: powdery mildew, brown rot, wilt, bacterial canker, bacterial blight, scab, tobacco mosaic virus (TMV I), tomato mosaic virus (TMV II), tomato infectious chlorosis virus (TICV), tomato spotted wilt virus (TSWV), etc.

### EXAMPLE 1

### Tests in a Controlled Environment

Plant materials used for tests were either branches obtained from the fields or trees grew in pots in a green house.

To test the effects of different glycerides on over winter pests such as San Jose Scale (SJS) or European red mite (ERM) on pome or stone fruit trees, stems from dormant ‘Delicious’ apple trees and ‘Feng Huang’ peach trees infested with SJS were collected and dipped in 2% of different glyceride emulsions with the untreated served as control. Stems were then trimmed to 25 cm long by removing from the base, put in a plate with the base submerging in 1-cm thick water, and incubated at 23 degree C for 2 weeks in a chamber. After incubation, 15 overwintering SJS (‘Black-cap’ stage of first instar) per stem were examined microscopically and assessed for viability. Apple or peach stems containing ERM eggs were collected in late dormant and treated as described above and incubated at 28 degree C., 80% relative humidity, and 16-hour light/8-hour dark cycle. Mite viability was evaluated by the following method. Stem segment were impaled onto insect pins that had been driven through 10×12 cm white posterboard containing a thin coat of sticky gel to trip newly hatched mites and facilitate their counting. Eggs were examined every 2 days until hatch was complete.

Plants growing in pot were inoculated with pests or pathogen and sprayed with 2% glyceride emulsions. Death rate of pests or number of lesions was evaluated one to three weeks after treatment.

Since mineral oil has been widely used for insect pest and disease control in agriculture (Taschenberg, 1952; Walsh et al., 2000), it was included in the experiments for comparison.

Under controlled conditions, all glycerides showed certain levels of pest and disease control (Tables 1, 2, 3, 4, 5, and 6). In general, the efficacy of glycerides in controlling pests and diseases were determined by their structure, in which monolaurin=lysophospholipids>other mono-glycerides=phospholipids>diglycerides>triglycerides and triglyceride type plant oils or animal oil>animal fats. Unsaturated diglycerides had higher activity in insect pest and disease control than saturated diglycerides. And mineral oil was more effective in insect pest or disease control than triglycerides but less effective than monoglycerides.

TABLE 1
Effects of 2% glyceride emulsion on pest survival (%) on ‘Delicious’
apple and ‘Qi Cheng’ orange trees. Data were collected in 1994-1998
season.

| | Apples | | | | | | |
|---|---|---|---|---|---|---|---|
| | Aph- | | Psyl- | White- | | Oranges | |
| Lipids | ids | Mites | la, | fly | SJS | Aphids | Mite |
| Control | 100a^{z} | 98a | 100a | 99a | 98a | 100a | 100a |
| Mono- | | | | | | | |
| acylglycerols | | | | | | | |
| Monolaurin | 34e | 0g | 35e | 31e | 0f | 31e | 39e |
| Lyso- | 31e | 0g | 31e | 27e | 0f | 28e | 34e |
| phospholipids | | | | | | | |
| Others | 43d | 0g | 62c | 51d | 5e | 47d | 51d |
| Diacylglycerols | | | | | | | |
| Saturated | 76b | 48c | 89b | 78b | 57b | 80ab | 73c |
| Monounsaturated | 72b | 23e | 92b | 68c | 36c | 77b | 70c |
| Polyunsaturated | 56c | 14f | 49d | 50d | 18d | 59c | 53d |
| Oils or fats | | | | | | | |
| Animal fat | 94a | 65b | 102a | 99a | 59b | 98a | 100a |
| Plant or fish oil | 75b | 34d | 85b | 71b | 36c | 89b | 86b |
| Mineral oil | 55c | 11f | 69c | 53d | 15d | 62c | 68c |

^{z}Mean separation in the same column for a particular pest by Duncan' new multiple range tests at p < 0.05.

TABLE 2
Effects of 2% glyceride emulsion on pest survival (%) on cotton, wheat, tomato, and
cabbages. Data were collected in 1994-1998 season.

| | Cotton | | Wheat | | Tomato | | Cabbage | |
|---|---|---|---|---|---|---|---|---|
| Lipids | Aphids | Whitefly | Aphids | Mites | Aphids | Mites | Aphids | Whitefly |
| Control | 100a^{z} | 100a | 100a | 100a | 100a | 100a | 100a | 100a |
| Monoacylglycerols | | | | | | | | |
| Monolaurin | 30e | 31f | 21e | 33e | 27e | 34e | 17e | 28f |
| Lysophospholipids | 34e | 30f | 19e | 29e | 28e | 29e | 24e | 35f |
| Others | 47d | 42e | 30d | 47d | 36d | 44d | 39d | 47e |
| Diacylglycerols | | | | | | | | |
| Saturated | 66b | 68c | 64b | 72c | 59b | 78b | 54c | 67c |
| Monounsaturated | 68b | 66c | 57b | 69c | 51b | 74b | 41d | 52d |
| Polyunsaturated | 56c | 57d | 42c | 43d | 43c | 55d | 38d | 43e |
| Oils or fats | | | | | | | | |
| Animal fat | 97a | 100a | 94a | 94a | 100a | 98a | 97a | 100a |
| Plant or fish oil | 74b | 79b | 65b | 76b | 65b | 79b | 64bc | 76b |
| Mineral oil | 59c | 53d | 59c | 65c | 61b | 58c | 52c | 49e |

^{z}Mean separation in the same column for a particular pest by Duncan' new multiple range tests at p < 0.05.

TABLE 3
Effects of 2% glyceride on disease severity (% of untreated control) on ‘Delicious’ apple,
‘Feng Hung’ peach, ‘Long Yan’ grape, and ‘Qi Cheng’ orange trees. Data were collected in
1994-1998 season.

| | Apples | | Peaches | | Grapes | | Oranges | |
|---|---|---|---|---|---|---|---|---|
| | | Powdery | Bacterial | Powdery | Leaf | Powdery | Brown | |
| Lipids | Scab | mildew | canker | mildew | spot | mildew | rot | Psorosis |
| Monoacylglycerols | | | | | | | | |
| Monolaurin | 14f^{z} | 18f | 24f | 13g | 37f | 16g | 34e | 32d |
| Lysophospholipids | 17ef | 15f | 29f | 19g | 41f | 19g | 39e | 35d |
| Others | 25e | 32de | 39e | 28f | 53e | 27f | 51d | 30d |
| Diacylglycerols | | | | | | | | |
| Saturated | 47c | 54c | 68c | 79b | 79c | 45d | 72c | 69b |
| Monounsaturated | 38d | 43d | 54d | 54d | 62d | 36e | 60d | 43c |
| Polyunsaturated | 36d | 37d | 42e | 41e | 48f | 23f | 51d | 38cd |
| Oils or fats | | | | | | | | |
| Animal fat | 84a | 82a | 100a | 94a | 100a | 82a | 97a | 100a |
| Plant or fish oil | 57b | 65b | 78b | 71bc | 79c | 67b | 81b | 74b |
| Mineral oil | 81a | 41d | 97a | 67c | 81bc | 51c | 100a | 97a |

^{z}Mean separation in the same column for a particular pest by Duncan' new multiple range tests at p < 0.05.

TABLE 4
Effects of 2% glyceride emulsion on disease severity (% of untreated
control) on cotton, and wheat. Data were collected in 1994-1998 season.

| | Cotton | | | |
|---|---|---|---|---|
| | Fusarium | Verticillium | Wheat | |
| Lipids | wilt | wilt | Barley strip | leaf scald |
| Mono- | | | | |
| acylglycerols | | | | |
| Monolaurin | 43e^{z} | 48d | 19f | 32f |
| Lyso- | 46e | 43d | 23f | 29f |
| phospholipids | | | | |
| Others | 59d | 62c | 34e | 46e |
| Diacylglycerols | | | | |
| Saturated | 79c | 71b | 54c | 67cd |
| Monounsaturated | 71c | 58cd | 46d | 59d |
| Polyunsaturated | 65d | 50d | 37e | 42e |
| Oils or fats | | | | |
| Animal fat | 97a | 89a | 84a | 94a |
| Plant or fish oil | 84b | 72b | 67b | 79b |
| Mineral oil | 99a | 91a | 69b | 81b |

^{z}Mean separation in the same column for a particular pest by Duncan' new multiple range tests at p < 0.05.

TABLE 5
Effects of 2% glyceride emulsion on disease severity (% of untreated con-
trol) on cabbage and potatoes. Data were collected in 1994-1998 season.

| | Cabbage | | Potato | |
|---|---|---|---|---|
| | Leaf- | Verticillium | Bacterial | |
| Lipids | spot | wilt | ring rot | Verticillium wilt |
| Mono- | | | | |
| acylglycerols | | | | |
| Monolaurin | 23f | 46de | 47e | 37e |
| Lyso- | 21f | 41e | 53e | 45d |
| phospholipids | | | | |
| Others | 28f | 47de | 51e | 52d |
| Diacylglycerols | | | | |
| Saturated | 59c | 74b | 82b | 73b |
| Monounsaturated | 50d | 66c | 73c | 57c |
| Polyunsaturated | 41e | 53d | 62d | 48d |
| Oils or fats | | | | |
| Animal fat | 93a | 92a | 99a | 91a |
| Plant or fish oil | 71b | 77b | 84b | 77b |
| Mineral oil | 93a | 94a | 97a | 93a |

^{z}Mean separation in the same column for a particular pest by Duncan' new multiple range tests at p < 0.05.

TABLE 6
Effects of 2% glyceride emulsion on disease severity (% of untreated
control) in tomatoes. Data were collected in 1994-1998 season.

| | Bac- | Verti- | Fusar- | | | | |
|---|---|---|---|---|---|---|---|
| | terial | cillium | ium | TMV | TMV | | |
| Lipids | Spot | wilt | wilt | (I) | (II) | TICV | TSWV |
| Mono- | | | | | | | |
| acylglycerols | | | | | | | |
| Monolaurin | 18f^{z} | 26f | 29f | 37e | 43d | 33f | 27e |
| Lyso- | 23ef | 29ef | 33f | 39e | 37d | 31f | 23e |
| phospholipids | | | | | | | |
| Others | 29e | 37e | 34f | 41e | 38d | 45e | 31e |
| Diacylglycerols | | | | | | | |
| Saturated | 54bc | 64c | 71c | 87b | 69b | 68c | 69c |
| Mono- | 50c | 52d | 59d | 69c | 58c | 59d | 53d |
| unsaturated | | | | | | | |
| Poly- | 38d | 42e | 47e | 54d | 59c | 51d | 48d |
| unsaturated | | | | | | | |
| Oils or fats | | | | | | | |
| Animal fats | 82a | 97a | 97a | 99a | 98a | 91a | 96a |
| Plant or fish oil | 61b | 82b | 81b | 81b | 74b | 78b | 80b |
| Mineral oil | 63b | 98a | 92a | 96a | 99a | 95a | 92a |

^{z}Mean separation in the same column for a particular pest by Duncan' new multiple range tests at p < 0.05.

### EXAMPLE 2

### Field Tests

The efficacy of different glycerides on disease and pest control was also tested under field conditions.

Dormant or delayed dormant spray at 2.5% or 5% of total lipid emulsion was applied to pome, stone, or other fruit trees. In experiments with other plants, fields with a history of pests or diseases were chosen and pests or diseases were monitored regularly during plant growth season. When pests were found on about 40% to 50% of plant population, or diseases found on 10% of plant population, emulsions at 1%, 2.5%, and 5% of total glycerides were sprayed with a low-pressure hand wand sprayers until run off. Death rate of pests, number of lesion, or foliage damage was evaluated from one to three weeks after treatment.

Emulsions at 1%-5% were effective in controlling pests and diseases in a concentration dependent manner (Table 7). Glycerides at 5% did not cause any observable injury to treated plants (data not shown).

TABLE 7
Effects of monolaurin or lysophospholipid emulsion on aphid survival (%)
and powder mildew (% of untreated control) on fruit trees and crops in the
fields. Data were collected in 1994-1998 season.

| | | | | | | | | |
|---|---|---|---|---|---|---|---|---|
| Lipid | Ap- | | Or- | Cot- | | Cab- | | |
| (%) | ple | Peach | ange | ton | Wheat | bage | Potato | Tomato |

Aphid survival

| | | | | | | | | |
|---|---|---|---|---|---|---|---|---|
| Control | 100a^{z} | 100a | 100a | 100a | 100a | 100a | 100a | 100a |
| 1% | 25b | 37b | 39b | 41b | 39b | 47b | 36b | 46b |
| 2.5% | 11c | 10c | 18c | 19c | 14c | 24c | 21c | 25c |
| 5% | 5d | 7d | 11d | 11d | 8d | 12d | 9d | 14d |

Powdery mildew

| | | | | | | | | |
|---|---|---|---|---|---|---|---|---|
| 1% | 41a | 39a | 46a | 49a | 37a | 37a | 52a | 48a |
| 2.5% | 14b | 10b | 24b | 22b | 18b | 19b | 27b | 29b |
| 5% | 4c | 12b | 10c | 17b | 6c | 4c | 13c | 17c |

^{z}Mean separation in the same column for a particular crop by Duncan' new multiple range tests at p < 0.05.

### Reference Ited

### U.S. Patent Documents

U.S. Pat. No. 4,002,775 January, 1977 Kabara 426/532

U.S. Pat. No. 4,997,851 March, 1991 Isaacs, et al. 514/558

U.S. Pat. No. 5,434,182 July, 1995 Isaacs et al. 514/546

U.S. Pat. No. 6,033,705 March, 2000 Isaacs 426/323

U.S. Pat. No. 5,366,995 November, 1994 Savage, et al. 514/558

U.S. Pat. No. 5,631,290 May, 1997 Almond et al. 514/560

U.S. Pat. No. 6,103,768 August, 2000 Savage, et al. 514/627

U.S. Pat. No. 6,136,856 October, 2000 Savage, et al. 514/552

U.S. Pat. No. 6,165,997 December, 2000 Cohen et al. 514/148

### Other references

Butler, G. D. Jr., Coudriet, D. L., Henneberry, T. J. 1988. Toxicity and repellency of soybean and cottonseed oils to the sweetpotato whitefly and the cotton aphid on cotton in greenhouse studies. Southwest. Entomol. 13:81-86.

Butler, G. D. Jr., Henneberry, T. J. 1989. Sweetpotato whitefly migration, population increase, and control on lettuce with cottonseed oil sprays. Southwest. Entomol. 14:287-293.

Butler, G. D. Jr., Henneberry, T. J. 1990. Cottonseed oil and safer insecticidal soap: Effects on cotton and vegetable pests and phytotoxicity. Southwest. Entomol. 15:257-264.

Butler, G. D. Jr., Henneberry, T. J. 1991 a. Effect of oil sprays on sweetpotato whitefly and phytotoxicity on watermelons, squash and cucumbers. Southwest. Entomol. 16:63-72.

Butler, G. D. Jr., Henneberry, T. J. 1991b. Sweetpotato whitefly control: Effect of tomato cultures and plant derived oils. Southwest. Entomol. 16:37-43.

Butler, G. D. Jr., Henneberry, T. J., Stansly, P. A., Schuster, D. J. 1993. Insecticidal effects of selected soaps, oils and detergents on the sweetpotato whitefly (Homoptera:Aleyrododae). Florida Entomology 76:161-167.

Butler, G. D. Jr., Puri, S. N., Henneberry, T. J. 1991. Plant derived oil and detergent solutions as control agents for Bemesia tabaci and aphis gossypii on cotton. Southwest. Entomol. 16:331-337.

Chase, A. R., Osborne, L. S. 1983. “Influence of an insecticidal soap on several foliar diseases of foliage plants”. Plant Disease 67:1021-1023.

Giga, D. P. and Munetsi, M. M. 1990. The effectiveness of vegetable and citrus oil as protectants of cowpeas against infestations by Callosobruchus rhodesianus (Pic). Plant Protection Quarterly 5:148-151.

Hesler, L. S. and Plapp, F. W. 1986. Uses of oil in insect control. Southwest Entomol. Suppl. No. 11:1-8.

Hill, J. and Schoonhoven, A. V. 1981. Effectiveness of vegetable oil fractions in controlling the Mexican bean weevil. J. Econ. Entomol. 74:478-479.

Kabara, J. J. 1993. Medium-chain fatty acids and esters. In Davidson P. M. and Branen, A. L. (eds) Antimicrobials in Foods. Marcel Dekker, Inc. New York. pp 307-342.

Locke, J. C. 1986. In vitro fungitoxicity of an ethanolic extract of neem seed on Rhizoctonia solani and Fusarium oxysporum. Phytopathology 76:1129-1132.

Locke, J. C. 1990. Activity of extracted neem seed oil against fungal plant pathogens P. 132-136. In Locke, J. C. and Lawson, R. H. (eds). “Neem's potential in pest management programs”. Proc. U.S. Dept. Agr. Neem Workshop. USDA ARS ARS-86.

Moline, H. E. and Locke, J. C. 1993. Comparing neem seed oil with calcium chloride and fungicides for controlling postharvest apple decay. HortScience 28, 719-720.

Northover, J. and Schneider K. E. 1991. Efficacy of canola and soybean oils against peach brown rot. Fungic. Nematicide Tests. 46:69.

Northover, J. and Schneider K. E. 1993. Activity of plant oils on diseases caused by Podosphaera leucotricha, Venneria inaequatis, and Albugo occidenratis. Plant Dis. 77:152-157.

Northover, J. and McFadden-Smith W. 1995. Control and epidemiology of Apiosporina morbosa of plum and cherry. Can. J. Plant Path. 17:57-68.

Pless, C. D., Deyton. D. E. and Sams, C. E. 1995. Control of San Jose scale, terrapin scale, and European red mite on dormant fruit trees with soybean oil. HortScience 30: 94-97.

Ran, P., Verma, R. S. and Singh, S. V. 1988. Vegetable oils as grain protectant against Sitophilus oryzea L. Farm Sci. J. 3:14-20.

Rock, G. C., Crabtree, K. W. 1987. Biological activity of petroleum and cottonseed oils against two tetranychid mite species and tortricid insect species found on apple. J. Agr. Entomol. 4:247-253.

Salas, J. 1985. Protection of maize seeds (Zea mays) against attack by Sitophilus oryzae through use of vegetable oils Agronomia-Tropical 35:13-18.

Salas, J. and Hernandez, G. 1985. Protection of pigeon pea seeds against attack by Acanthoscelides obtectus and Callosobruchus maculatus through the use of vegetable oils. Agronomia-Tripical 35:19-27.

Taschenberg, E. F. 1952. Evaluation of petroleum and vegetable oils on grape berry moth eggs. Econ. Entomol. 45:85-91.

Walsh, D. B., Zalom, F. G., Grove G. G. 2000. Petroleum spray oils: An airblast from the past, with a slick future. Good Fruit Grower 51:45-48.

U.S. EPA. 1992a. R.E.D. Facts: soap salts. EPA-738-F-92-013.

U.S. EPA. 1992b. R.E.D. Facts: flower and vegetable oils. EPA-738-F-93-027.

The following is claimed:

1. A composition for controlling diseases and pests, the composition comprising:
polar glycerides, surfactant, and water.

2. The composition of claim 1, wherein the polar glycerides comprise compounds selected from the group consisting of mono glycerides, di glycerides, phospholipids, lysophosphlipids, glycolipids, lysoglycolipids, and their derivative, or the mixture of said polar lipids with any ratio.

3. The composition of claim 1, wherein the surfactant is chosen from the group comprising siloxanes, polysiloxanes, polyoxyethylene ethers, polyoxyethylenesorbitan, alkylaryl polyoxyethylene glycols and alcohol, or any other nonionic surfactants.

4. The composition of claim 1, wherein the polar glyceride comprises from approximately 1% up to 90% of the total emulsion, by volume; the surfactant comprises from approximately 0 to 30% of the total emulsion, by volume; and water comprises from 1 to 99.5% of the total emulsion, by volume.

5. The composition of claim 1, wherein the polar glycerides, surfactant, and water are present preferably in a volume to volume ratio of approximately 5:1:4.

6. A method for controlling diseases and pests on plants, the method including the steps of:
a) mixing polar glycerides, surfactant, and water to form an emulsion.
b) diluting said emulsion with water to appropriate concentration.
c) applying an effective amount of the diluted emulsion on plants.

7. The method of claim 6, wherein the step of mixing the polar glycerides, neutral glycerides, and surfactant further includes the step of heating the resultant mixture to 50-95° C.

8. The method of claim 6, wherein the step of mixing the polar glycerides and surfactant further includes the step of adding preheated water (50-95° C.) to the mixture.

9. The composition and method of claim 6, wherein the effective amount of emulsion applied to trees includes a concentration of active ingredients (including emulsifier) at 0.5% to 10%, preferably at 1% to 3%.

10. The composition and method of claim 8, wherein the plants include agricultural crops, fruit trees, vegetables, forage, bedding plants, nursery plants, ornamentals, houseplants, and turf grasses.

11. The composition and method of claim 8, wherein the pests include aphids, mites, pear psylla, scales, whiteflies, or other soft body pests or eggs of other pests.

12. The composition and method of claim 8, wherein the diseases include diseases caused by fungus, bacteria, and virus such as powdery mildew, brown rot, fire blight, wilt, bacterial canker, bacterial blight, scab, tobacco mosaic virus (TMV I), tomato mosaic virus (TMV II), tomato infectious chlorosis virus (TICV), tomato spotted wilt virus (TSWV).
